SEQUENCE LISTING (10 sequences)

SEQ ID NO: 1 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - DNA, 4584 bp, Homo sapiens
  source 1..4584: mol_type = other DNA
cttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaagaaagaaaggaaaataaaaaatgatagttgccattattaggatttaatatatatccagtgctttgcaagtgctctgcgcaccttgtctcactccatcctgacaataatcctgggaggtgtgtgcaattactacgactactctcttttttatagatcattaaattcagaactaaggagttaagtaacttgtccaagttgttcacacagtgaagggaggggccaagatatgatggctgggagtctaattgcagttccctgagccatgtgcctttctcttcactgaggactgccccattcttgagtgccaaacgtcactagtaacagggtgtgcctagataatttatgatccaaactgagtcagtttggaaagtgaaagggaaacttacatataatccctccgggacaatgagcaaaaactaggactgtccccagacaaatgtgaacatacatatcatcacttaaattaaaatggctatgagaaagaaagagggggagaaacagtcttgcgggtgtgaagtcccatgaccagccatgtcaaaagaaggtaaagaagtcaagaaaaagccatgaagcccatttggtttcatttttctgaaaataggctcaagagggaataaattagaaactcacaatttctcttgtttgttaccaagacagtgattctcttgctgctaccacccaactgcatccgtccatgatctcagaggaaactgtcgctgaccctggacatgggtacgtttgacgagtgagaggaggcatgacccctcccatgtgtatagacactaccccaacctaaattcatccctaaattgtcccaagttctccagcaatagaggctgccacaaacttcagggagaaagagttacaagtacatgcaatgagtgaactgactgtggctacaatcttgaagatatacggaagagacgtattattaatgcttgacatatatcatcttgcctttcttggtctagactgacttctaatgactaactcaaagtcaaggcaactgagtaatgtcagctcagcaaagtgcagcaaacccatctcccacaggcctccaaaccctggctgttcacagaaccacaaagggcagatgctgcacagaaaactagagaaggggtcataggttcatggttttgtttgagatttgttgctactgtttttctgttttgaattttcttctttgttctgtttttactttatttagggggactaggtgtttctgatattttagttttcttgtttgttttgttttgtgttgtctgtgaatggggttttaactgtggatgaatggaccttatctgttggcttaaaggactggtaagatcagaccatcttattcttcaggtgaatgttttactttccaaagtgctctcctctgcaccagcagtaataaatacaatgccataatcccttaggtttgcctagtgcttttgcaattttcaaagcacttccataagcattccttccacctccttgataggcatttatggaaagcctgctacatgtcaatcatactgttaggcacaggggacctaaagacacataaaaggatggcattctgcctcataaattgcaaaacctaatgaaagtgactgcttggtaaacaaattattattatattataaaatgctataaaagagccatattgaaagtgccctgttggagacagggcaaatgccacaaaaatgatgtaaatttacatggaggaaaagtagaatctgcctggtttgtaggcagcagaagacatttttcatcagtgggcaggtgttctttaccttttgtagaaatgggagtcaagtctcaaataggaggctccacaaaatctcatgccaggtctctgataccttattcacagaagttctttgaagtatttattgttattttctttgacttatgggaaaactgggacacaggaagacaggtaaattacccaacctcacacgttaagtcagaactgggagccataattttgtatccctggtataaatagacaatctcttgaagaaatgaagagatgaccatagaaaaacatcgagatatctccagctctaaaatcctttgtttcaatgttgtttggcatatgttatctttggaatttagtgtctgagcctctgtctgttactgtagtatttaaaatgcatgtattataatcatataatcataactgctgttaattcttgattatatacctagggacaatgtgtaatgtaagattactaattggttctgcccaatctcctttcagattttattaggaaaaaaaaataaacctcctgatcggagacaatgtattaatcagaagtgtaaactgccagttctatatagcatgaaatgaaaagacagctaatttggtccaacaaacatgactgggtctagggcacccaggctgattcagctgatttcctaccagcctttgcctcttccttcaatgtggtttccatgggaatttgcttcagaaaagccaagtatgggctgttcagaggtgcacacctgcattttcttagctcttctagaggggctaagagacttggtacgggccaggaagaatatgtggcagagctcctggaaatgatgcagattaggtggcatttttgtcagctctgtggtttattgttgggactattctttaaaatatccattgttcactacagtgaagatctctgatttaaccgtgtactatccacatgcattacaaacatttcgcagagctgcttagtatataagcgtacaatgtatgtaataaccatctcatatttaattaaatggtatagaagaaca

SEQ ID NO: 3 - DNA, 190 bp, Homo sapiens
  source 1..190: mol_type = other DNA
ctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgt

SEQ ID NO: 4 - DNA, 87 bp, Homo sapiens
  source 1..87: mol_type = other DNA
cttcctccctccctcccttcctcttactctcattcatttcatacacactggctcacacatctactctctctctctatctctctcaga

SEQ ID NO: 5 - DNA, 523 bp, Homo sapiens
  source 1..523: mol_type = other DNA
ctcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 6 - DNA, 616 bp, Woodchuck hepatitis virus
  source 1..616: mol_type = other DNA
gtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacc

SEQ ID NO: 7 - DNA, 190 bp, unidentified
  source 1..190: mol_type = other DNA
gtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgg

SEQ ID NO: 8 - DNA, 36 bp, synthetic construct
  source 1..36: mol_type = other DNA
aagggagacatctagtgatataagtgtgaactacac

SEQ ID NO: 9 - DNA, 9318 bp, synthetic construct
  source 1..9318: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatgtatacgtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgggatatctcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggtaccctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgagtcgactttaagaccaatgacttacaaggcagctgtagatcttagccactttttaaaagaaaaggggggactggaagggctaattcactcccaacgaagacaagatctgctttttgctgtgtagttcacacttatatcactagatgtctcccttactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggctagcaaacaaaagatctagaaagaaacaaacccgggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggaaagaaccagctggggctcgagatccactagttctagcctcgaggctagagcggccgccaccgcggtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 10 - DNA, 9146 bp, synthetic construct
  source 1..9146: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatgtatacgtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgggatatccaagacctgagtgaggaaagagagaaggcactgttgttcccccacaatctctagaaggactggtgacgcatgtcatttttctttgagctccccacactttaggcttctctctgaggtcttttcagcatctcctgccccacccaagagtaagcacccctgtggaaatccccagaacaaaccagcacatgctgtaccaaaatattgtgtcttggctttgttcttttacatcttcacaactaaaggaaagagatacaatcaaaatgatagtccacttaaatgataaacaagagaatttaaagagacataacccagttactcacccatgaagtgacaatctgttagcagtttagggtaccctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgagtcgactttaagaccaatgacttacaaggcagctgtagatcttagccactttttaaaagaaaaggggggactggaagggctaattcactcccaacgaagacaagatctgctttttgctgtgtagttcacacttatatcactagatgtctcccttactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggctagcaaacaaaagatctagaaagaaacaaacccgggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggaaagaaccagctggggctcgagatccactagttctagcctcgaggctagagcggccgccaccgcggtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

(19) United States
(12) Patent Application Publication
(10) Pub. No.: US 2024/0417444 A1
(43) Pub. Date: Dec. 19, 2024

(54) COMPOSITIONS AND METHODS FOR TREATING INTERLEUKIN 7 RECEPTOR DEFICIENCY

(71) Applicant: The Children's Hospital of Philadelphia, Philadelphia, PA (US)

(72) Inventors: Stefano Rivella, Philadelphia, PA (US); Laura Breda, Philadelphia, PA (US); Michael P. Triebwasser, Ann Arbor, MI (US)

(21) Appl. No.: 18/700,137

(22) PCT Filed: Nov. 3, 2022
(86) PCT No.: PCT/US2022/079207

§ 371 Date: Apr. 10, 2024

Related U.S. Application Data
(60) Provisional application No. 63/275,543, filed on Nov. 4, 2021.

(51) Int. Cl. C07K 14/715; A61K 38/00; A61K 48/00; A61P 37/04; C12N 15/86

(52) U.S. Cl. CPC C07K 14/7155; A61K 48/005; A61P 37/04; C12N 15/86; A61K 38/00; C12N 2740/15042; C12N 2740/15043; C12N 2830/50

(57) ABSTRACT

Lentiviral vectors comprising a nucleic acid sequence encoding human IL-7R, for producing IL-7R and treating IL-7R deficiency are disclosed. Compositions, viral particles, and cells comprising the vector are further disclosed. Methods for use of these vectors or cells comprising these vectors for treating an IL-7R deficiency, such as severe combined immunodeficiency, are also provided.

This application claims priority under 35 U.S.C. § 119 (e) to U.S. Provisional Patent Application No. 63/275,543, filed Nov. 4, 2021. The foregoing application is incorporated by reference herein.

### FIELD OF THE INVENTION

The present invention relates to the field of immunodeficiency. More specifically, the invention provides compositions and methods for the treatment of interleukin 7 (IL-7) receptor (IL-7R) deficiency.

### BACKGROUND OF THE INVENTION

Several publications and patent documents are cited throughout the specification in order to describe the state of the art to which this invention pertains. Each of these citations is incorporated herein by reference as though set forth in full.

In humans, interleukin 7 (IL-7) receptor (IL-7R) deficiency causes approximately 10% of cases of severe combined immunodeficiency (SCID). IL-7R deficient SCID is a T-B+NK+ SCID (lacks T cells) and is caused by autosomal recessive deficiency of the IL-7R alpha chain gene (IL7R). IL-7R is a heterodimeric receptor comprised of the alpha chain and the IL-2 receptor common gamma chain (IL2RG). In both mouse and human, IL-7R is a marker of the common lymphoid progenitor cell, and IL-7 signaling leads to STAT5 phosphorylation and proliferation of developing T and B cells. Mice lacking IL7R, Il7r−/−, lack both T and B cells (Peschon, et al., J. Exp. Med. (1994) 180 (5): 1955-60). T cells do not progress to TCR beta chain rearrangement and B cell development is halted at the pre-pro-B cell stage in the absence of IL-7 signaling. Similar to the mouse, IL-7 signaling in humans is required for T cell receptor beta gene rearrangement and T cell maintenance. However, humans lacking IL-7R can develop B cells as human pro-B cells can undergo VDJ recombination without IL-7R.

Currently available therapies for IL-7R deficiency are limited-only by allogeneic bone marrow transplantation. Thus, there is an ongoing and unmet need for improved compositions and methods for treating IL-7R deficiency.

### SUMMARY OF THE INVENTION

In accordance with one aspect of the instant invention, nucleic acid molecules, particularly vectors (e.g., viral vectors such as lentiviral vectors), are provided. In a particular embodiment, the vector comprises a nucleic acid molecule comprising: i) a 5′ long terminal repeat (LTR) and a 3′ LTR, such as a lentiviral LTR, optionally wherein at least one of the LTR (e.g., the 3′LTR) is self-inactivating; and ii) a sequence encoding a IL-7R, particularly human IL-7R, optionally including at least part of the 5′UTR of the IL-7R gene (e.g., including the promoter and/or enhancer). In certain embodiments, the nucleic acid molecule further comprises one or more of: i) a promoter (e.g., a constitutive promoter (e.g., PGK promoter)); ii) a polyadenylation signal (e.g., a strong bovine growth hormone poly A tail (e.g., inserted after the WPRE region, if present)); iii) an enhancer (e.g., an IL-7R enhancer, particularly DHS1 and/or DHS2); iv) an insulator element (e.g., an ankyrin insulator element (Ank) and/or foamy virus insulator); and v) a Woodchuck Post-Regulatory Element (WPRE) (e.g., configured such that the WPRE does not integrate into a target genome; e.g., outside of the LTRs). The instant invention also encompasses cells (e.g., bone marrow cells or hematopoietic stem cells or hematopoietic progenitor cells) comprising the vector (e.g., lentiviral vector) of the instant invention. Compositions comprising the vector (e.g., lentiviral vector) are also encompassed by the instant invention. The compositions may further comprise a carrier such as a pharmaceutically acceptable carrier.

In accordance with another aspect of the instant invention, methods of increasing expression of IL-7R (e.g., in vitro or in vivo) and methods of inhibiting, treating, and/or preventing IL-7R deficiency (e.g., IL-7R SCID) in a subject are provided. In a particular embodiment, the method comprises administering a viral vector of the instant invention (e.g., viral particles) to a subject in need thereof. In a particular embodiment, the method comprises an ex vivo therapy (e.g., autologous bone marrow cells or hematopoietic stem cells) utilizing a viral vector of the instant invention. The viral vector may be in a composition with a pharmaceutically acceptable carrier.

### BRIEF DESCRIPTIONS OF THE DRAWINGS

FIG. 1 provides schematics of the IL-7R viral vectors.

FIG. 2A provides a graph of the percent of peripheral blood leukocytes in wild type (WT) mice or IL-7R KO mice. ***p<2×10⁻³. ****p<5×10⁻⁴. FIG. 2B provides a graph showing the number of white blood cells (WBC) and absolute lymphocyte and neutrophil counts in wild type and IL-7R KO mice. **p<0.05.

FIG. 3 provides a graph of the vector copy number (VCN) in wild-type mice, untreated IL-7R KO mice, IL-7R KO mice treated with vPGK_DHS1 or vDHS1 transplanted bone marrow cells at 1 or 2 months after transplant.

FIGS. 4A, 4B, 4C, 4D, 4E, and 4F provide graphs of the white blood cells, absolute neutrophil count, absolute neutrophil count, absolute monocyte count, hemoglobin, and platelet count, respectively, in wild-type mice, untreated IL-7R KO mice, IL-7R KO mice treated with vPGK_DHS1 or vDHS1 transplanted bone marrow cells at 1, 2, or 3 months after transplant.

FIG. 5A provides graphs showing the percentage of CD45+ cells that are T cells or B cells in wild-type mice, untreated IL-7R KO mice, IL-7R KO mice treated with vPGK_DHS1 or vDHS1 transplanted bone marrow cells at 1 or 2 months after transplant. FIG. 5B provides a graph showing the percentage of CD45+ cells that are Gr1+ cells in wild-type mice, untreated IL-7R KO mice, IL-7R KO mice treated with vPGK_DHS1 or vDHS1 transplanted bone marrow cells at 1 or 2 months after transplant.

FIGS. 6A-6C provide a nucleotide sequence (SEQ ID NO: 9) of a vector comprising a PGK promoter, DHS1, and IL7R cDNA.

FIGS. 7A-7C provide a nucleotide sequence (SEQ ID NO: 10) of a vector comprising a PGK promoter, DHS1, DHS2, and IL7R cDNA.

### DETAILED DESCRIPTION OF THE INVENTION

Severe combined immunodeficiency (SCID) describes a group of life-threatening primary immunodeficiencies that have in common a failure of T lymphocyte production. Patients with SCID are therefore susceptible to infections from common bacteria, viruses, fungi, and opportunistic organisms; their long-term survival requires immune reconstitution, such as by successful hematopoietic stem cell transplantation (HSCT) or enzyme-replacement therapy (in the case of adenosine deaminase deficiency). T cell development and proliferation depend upon cytokine signaling, and SCID results from mutations of the genes encoding the common gamma chain (γc) of the receptors for interleukins (IL)-2, -4, -7, -9, -15, and -21; the Jak3 signaling kinase; or the IL-7 receptor a chain (IL-7Rα). Mutations in the X-linked IL2RG gene encoding γc affect males and cause roughly half of all cases of SCID. Mutations of IL7R, encoded on chromosome 5p13, account for at least 10% of cases of SCID and occur in both males and females (˜1 in 10⁶ individuals).

A retroviral vector (mouse stem cell virus, MSCV) has been used to rescue murine IL-7R deficiency wherein the vector contained the MSCV retroviral promoter or EF1α promoter, and the murine Il7r gene (Jiang, et al, Gene Therapy (2005) 12(24):1761-8). This strategy did restore T cells and had variable restoration of B cells. However, the MCSV retroviral-based gene addition of Il7r led to a myeloproliferative condition with significant neutrophilia and splenomegaly, ostensibly due to IL7 signaling in myeloid cells. Transduced bone marrow cells formed myeloid progenitors in response to IL-7 in vitro.

Herein, a novel gene therapy for IL-7R deficient SCID is provided that utilizes the human IL7R gene (e.g., human IL7R cDNA). To prevent lineage skewing, ectopic expression of IL7R in non-lymphoid cells was limited by utilizing the endogenous control elements such as the enhancers and promoters of IL7R. These sequences were identified as sites of high sequence conservation across species and DNA accessibility/hypersensitivity (DHS) in human lymphocytes. Use of the endogenous control elements will also provide the minimal IL-7R expression required for proper development. These sequences were tested alone or in combination with the constitutive phosphoglycerate kinase (PGK) promoter in VSV-G pseudotyped lentiviral vectors (LV): vPGK_DHS_hIL7R and vDHS_hIL7R. Herein, data is provided showing the ability of the human IL-7R protein to functionally replace the murine IL-7R protein and the ability of IL7R gene addition to rescue the murine Il7r^{-/-} immunodeficient phenotype in vivo. The transduced cells will have a competitive advantage and repopulate the thymus and B-cell compartment.

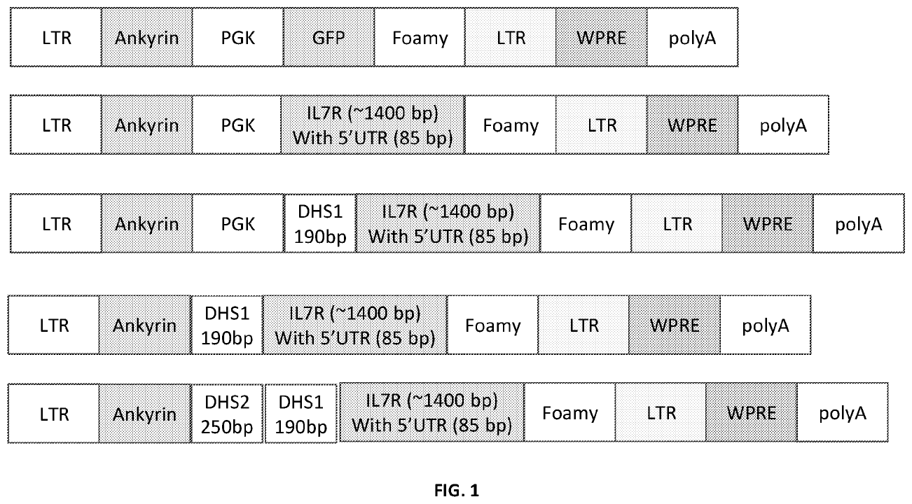

FIG. 1 provides schematics of five different vectors. First, a vector expressing the GFP gene using the human phosphoglycerate kinase (PGK) promoter was generated. Second, a vector expressing the IL7R gene using the PGK promoter and the 5′UTR/promoter from the IL7R gene was generated. Third, a vector expressing the IL7R gene using the PGK promoter, the 5′UTR/promoter from the IL7R gene, and the hypersensitive site DHS1/enhancer from the IL7R locus was generated. The DHS1 enhancer and/or IL7R promoter can facilitate the expression of the IL7R gene in the tissue of interest. Fourth, a vector expressing the IL7R gene using the 5′UTR from the IL7R gene and the hypersensitive site DHS1/enhancer from the IL7R locus was generated. The DHS1 enhancer and/or IL7R promoter allows the expression of the IL7R gene only in the tissue of interest. Fifth, a vector expressing the IL7R gene using the 5′UTR from the IL7R gene and the hypersensitive sites DHS1 and DHS2 (enhancers) from the IL7R locus was generated. The DHS1 and DHS2 enhancers and/or IL7R promoter allow the expression of the IL7R gene only in the tissue of interest.

Herein, nucleic acid molecules and vectors (e.g., viral vectors) for increasing IL-7R expression/production and/or the inhibition, prevention, and/or or treatment of IL-7R deficiency (e.g., IL-7R SCID) are provided. Generally, the nucleic acids of the instant invention will be a vector, particularly a viral vector. In certain embodiments, the viral vector comprises: i) a 5′ long terminal repeat (LTR) and a 3′ LTR (particularly, at least one of the LTR (at least the 3′LTR) is self-inactivating; a self-inactivating LTR comprises a deletion or mutation relative to its native sequence that results in it being replication incompetent); and ii) a sequence encoding a IL-7R (particularly including at least part of the 5′UTR of the IL-7R gene (e.g., including the promoter and/or enhancers)). In certain embodiments, the vector further comprises one or more of:
i) at least one promoter (e.g., a constitutive promoter (e.g., PGK promoter); e.g., operably linked or controlling expression of the IL7-R);
ii) at least one polyadenylation signal (e.g., a strong bovine growth hormone poly A tail (e.g., inserted after the WPRE region, if present) increases lentiviral titers (Zaiss, et al. (2002) J. Virol., 76(14):7209-19));
iii) an enhancer (e.g., an IL-7R enhancer, particularly DHS1 and/or DHS2; e.g., operably linked or controlling expression of the IL-7R);
iv) at least one insulator element (e.g., an ankyrin insulator element (Ank) and/or foamy virus insulator; particularly, the insulator is adjacent to or within an LTR); and
v) a Woodchuck Post-Regulatory Element (WPRE) (e.g., configured such that the WPRE does not integrate into a target genome; particularly 3′ of the 3′ LTR; e.g. Woodchuck Hepatitis Virus Postranscriptional Regulatory Element).

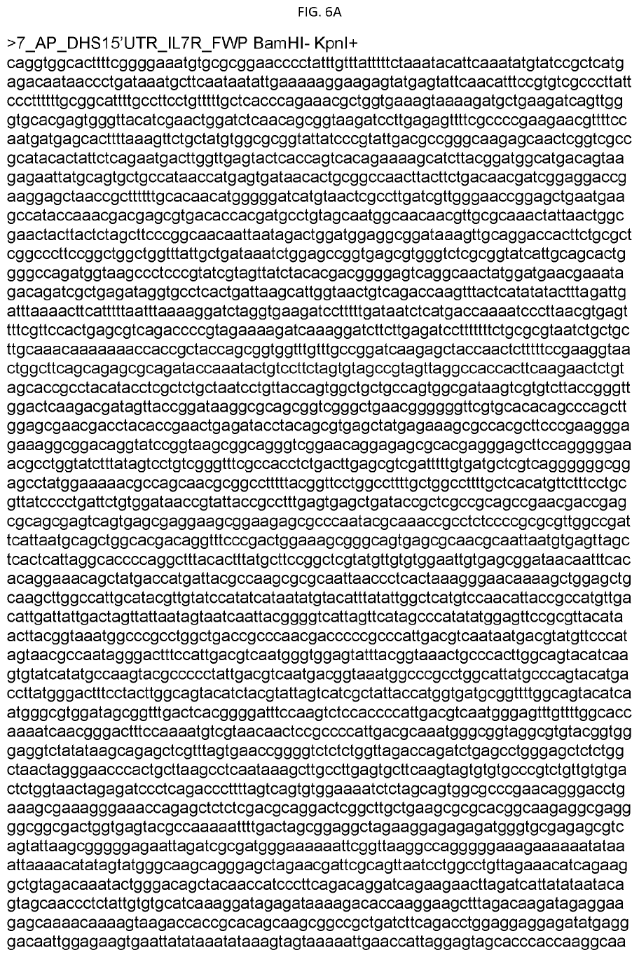

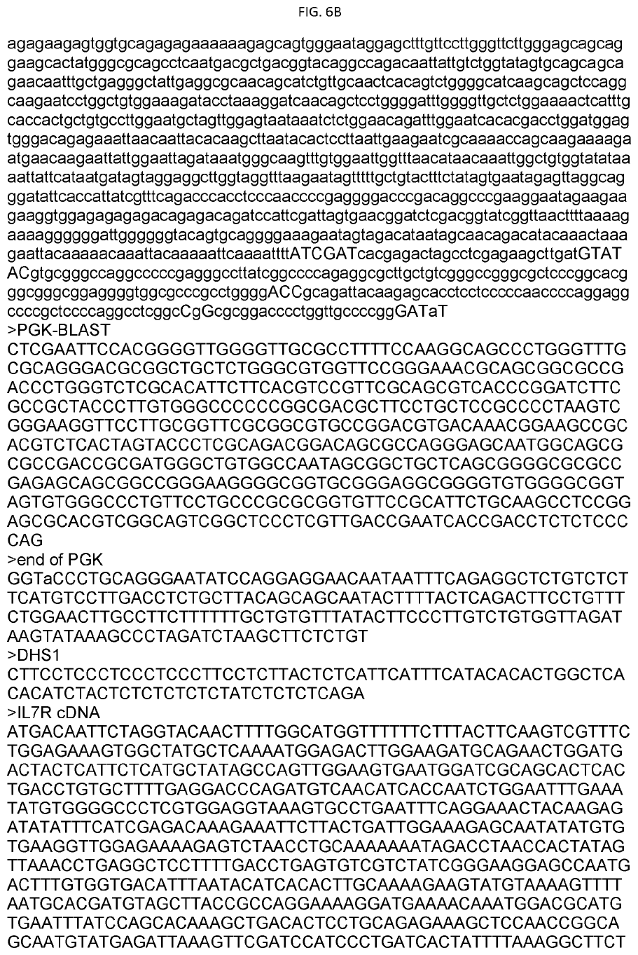

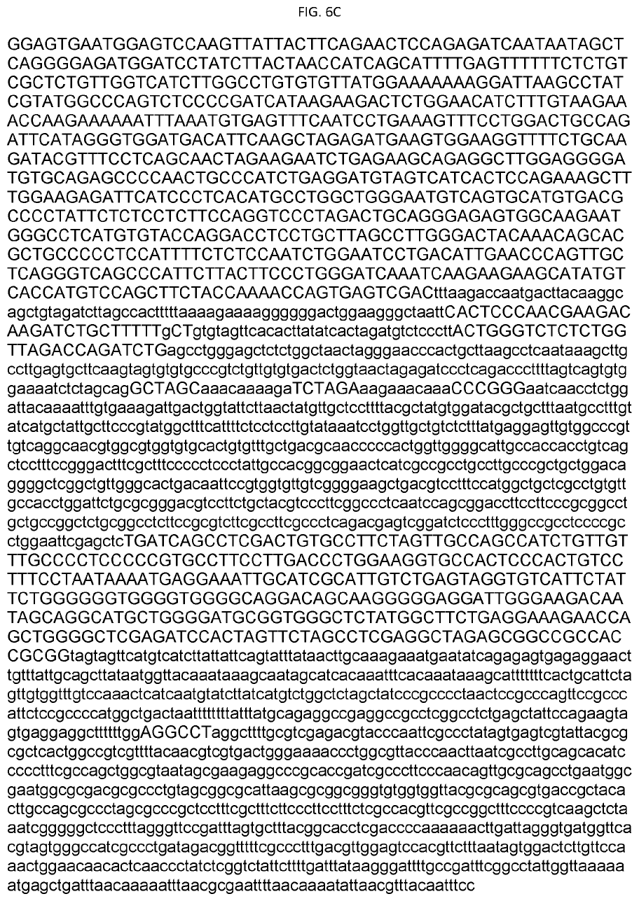

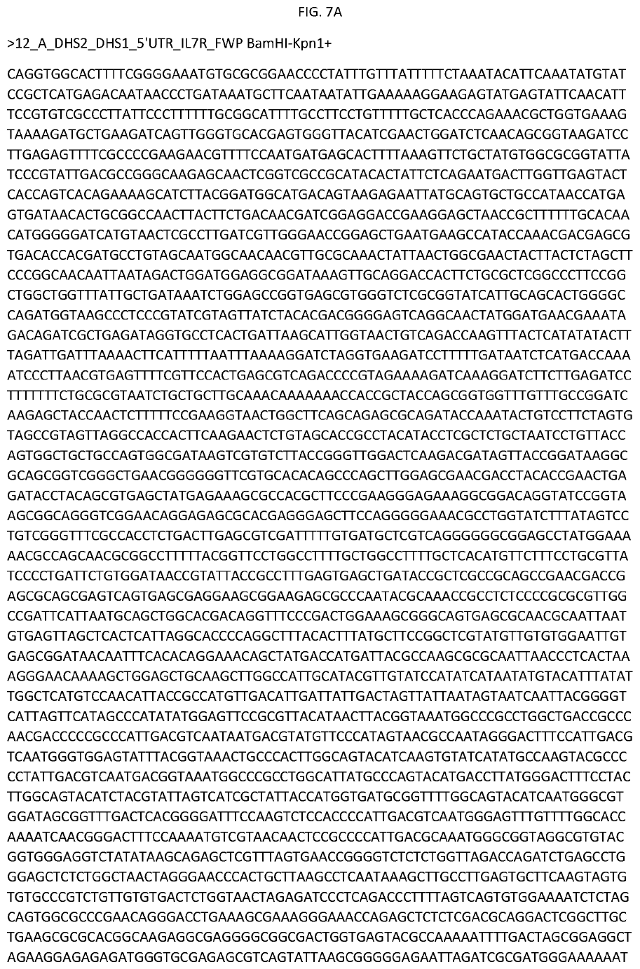

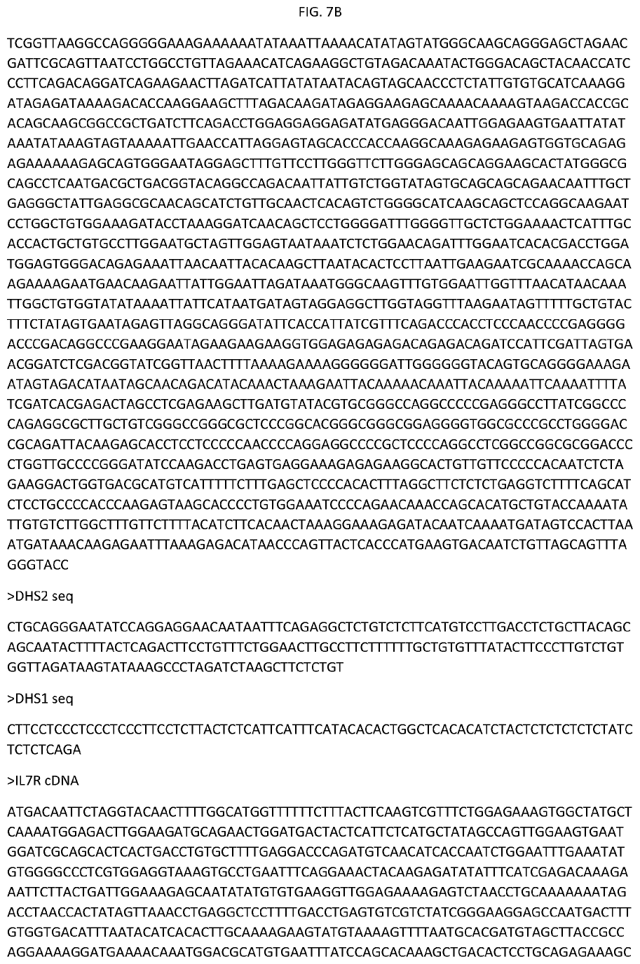

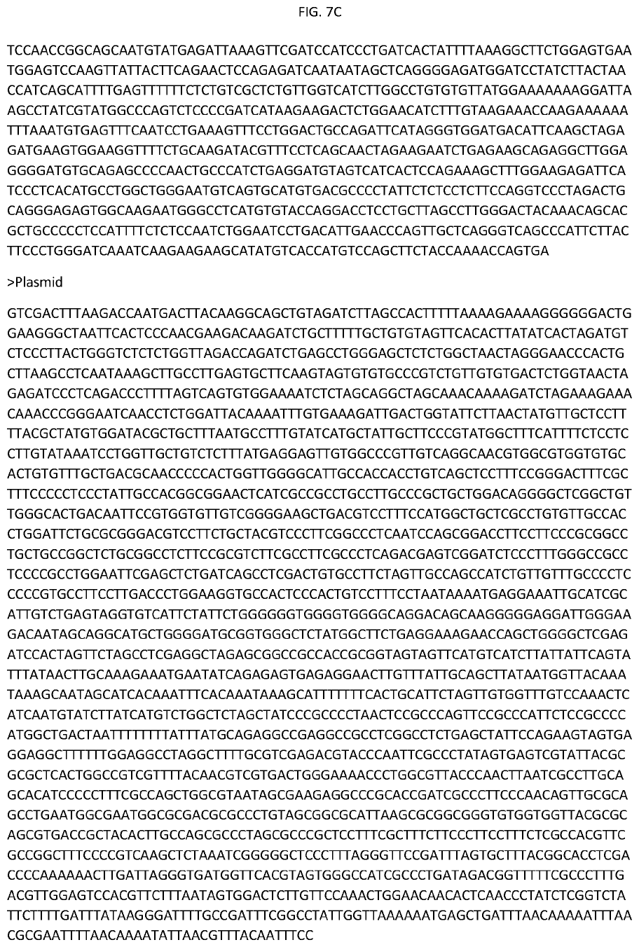

FIGS. 6 and 7 as well as U.S. Patent Application Publication 2018/0008725; WO 2019/213011; and WO 2020/264488 (these applications are incorporated by reference herein in their entirety), provide viral vectors as well as sequences for certain of the above elements.

In certain embodiments, the viral vector comprises (particularly from 5′ to 3′): i) a 5′ long terminal repeat (LTR); ii) an insulator element (e.g., an ankyrin insulator element (Ank)); iii) at least one promoter (e.g., a constitutive promoter (e.g., PGK promoter)); iv) a sequence encoding a IL-7R (e.g., including at least part of the 5′UTR of the IL-7R gene (e.g., including the IL-7R promoter)); v) an insulator element (optionally within the 3′LTR; e.g., a foamy virus insulator); vi) a 3′ LTR (e.g., a self-inactivating 3′ LTR), vii) a WPRE; and viii) a polyadenylation signal (e.g., a strong bovine growth hormone poly A tail).

In certain embodiments, the viral vector comprises (particularly from 5′ to 3′): i) a 5′ long terminal repeat (LTR); ii) an insulator element (e.g., an ankyrin insulator element (Ank)); iii) at least one promoter (e.g., a constitutive promoter (e.g., PGK promoter)); iv) a IL-7R enhancer (e.g., DHS1 and/or DHS2); v) a sequence encoding a IL-7R (e.g., including at least part of the 5′UTR of the IL-7R gene (e.g., including the IL-7R promoter)); vi) an insulator element (optionally within the 3′LTR; e.g., a foamy virus insulator); vii) a 3′ LTR (e.g., a self-inactivating 3′ LTR), viii) a WPRE; and ix) a polyadenylation signal (e.g., a strong bovine growth hormone poly A tail).

In certain embodiments, the viral vector comprises (particularly from 5′ to 3′): i) a 5′ long terminal repeat (LTR); ii) an insulator element (e.g., an ankyrin insulator element (Ank)); iii) a IL-7R enhancer (e.g., DHS1 and/or DHS2); iv) a sequence encoding a IL-7R (e.g., including at least part of the 5′UTR of the IL-7R gene (e.g., including the IL-7R promoter)); v) an insulator element (optionally within the 3′LTR; e.g., a foamy virus insulator); vi) a 3′ LTR (e.g., a self-inactivating 3′ LTR), vii) a WPRE; and viii) a polyadenylation signal (e.g., a strong bovine growth hormone polyA tail).

Autologous bone marrow cell or hematopoietic stem cell (HSC) gene therapy based on lentiviral gene addition, performed with reduced toxicity mono-agent conditioning, is an attractive curative cell therapy approach for IL-7R deficiency, as it eliminates risks of alloimmune complications, and has been associated with tolerable rates of organ toxicity when applied to patients with hemoglobin disorders (Thompson, et al., N. Engl. J. Med. (2018) 378(16):1479-1493). Thus, lentiviral gene correction is a very promising curative modality for these patients.

Viral vectors of the instant invention include, for example, retroviruses and lentiviruses. In a particular embodiment, the viral vector is a lentiviral vector. The nucleic acid molecule, vector, or viral vector of the instant invention may comprise one or more (or all) of the modifications listed below.

First, in certain embodiments of the instant invention, the IL-7R of the instant invention is human. Examples of amino acid and nucleotide sequences of IL-7R (CD127 or IL-7Rα) are provided in GenBank Gene ID: 3575 and GenBank Accession Nos. NM_002185.5 and NP_002176.2. An example of an amino acid sequence of human IL-7R is (SEQ ID NO: 1):

| | |
|---|---|
| 1 | MTILGTTFGM VESLLQVVSG ESGYAQNGDL EDAELDDYSF SCYSQLEVNG |
| 51 | SQHSLTCAFE DPDVNITNLE FEICGALVEV KCLNFRKLQE IYFIETKKEL |
| 101 | LIGKSNICVK VGEKSLTCKK IDLTTIVKPE APFDLSVVYR EGANDFVVTF |
| 151 | NTSHLQKKYV KVLMHDVAYR QEKDENKWTH VNLSSTKLTL LQRKLQPAAM |
| 201 | YEIKVRSIPD HYFKGFWSEW SPSYYFRTPE INNSSGEMDP ILLTISILSE |
| 251 | FSVALLVILA CVLWKKRIKP IVWPSLPDHK KTLEHLCKKP RKNLNVSENP |
| 301 | ESFLDCQIHR VDDIQARDEV EGFLQDTFPQ QLEESEKQRL GGDVQSPNCP |
| 351 | SEDVVITPES FGRDSSLTCL AGNVSACDAP ILSSSRSLDC RESGKNGPHV |
| 401 | YQDLLLSLGT TNSTLPPPFS LQSGILTLNP VAQGQPILTS LGSNQEEAYV |
| 451 | TMSSFYQNQ |

Amino acids 1-20 are a signal peptide and amino acids 21-459 is the mature protein. The IL-7R of the instant invention may contain the signal peptide or it may be absent. An example of a nucleotide sequence encoding human IL-7R is (SEQ ID NO: 2):

| | |
|---|---|
| 1 | cttcctccct ccctcccttc ctcttactct cattcatttc atacacactg gctcacacat |
| 61 | ctactctctc tctctatctc tctcagaatg acaattctag gtacaacttt tggcatggtt |
| 121 | ttttctttac ttcaagtcgt ttctggagaa agtggctatg ctcaaaatgg agacttggaa |
| 181 | gatgcagaac tggatgacta ctcattctca tgctatagcc agttggaagt gaatggatcg |
| 241 | cagcactcac tgacctgtgc ttttgaggac ccagatgtca acatcaccaa tctggaattt |
| 301 | gaaatatgtg gggccctcgt ggaggtaaag tgcctgaatt tcaggaaact acaagagata |
| 361 | tatttcatcg agacaaagaa attcttactg attggaaaga gcaatatatg tgtgaaggtt |
| 421 | ggagaaaaga gtctaacctg caaaaaaata gacctaacca ctatagttaa acctgaggct |
| 481 | ccttttgacc tgagtgtcgt ctatcgggaa ggagccaatg actttgtggt gacatttaat |
| 541 | acatcacact tgcaaaagaa gtatgtaaaa gttttaatgc acgatgtagc ttaccgccag |
| 601 | gaaaaggatg aaaacaaatg gacgcatgtg aatttatcca gcacaaagct gacactcctg |
| 661 | cagagaaagc tccaaccggc agcaatgtat gagattaaag ttcgatccat ccctgatcac |
| 721 | tattttaaag gcttctggag tgaatggagt ccaagttatt acttcagaac tccagagatc |
| 781 | aataatagct caggggagat ggatcctatc ttactaacca tcagcatttt gagttttttc |
| 841 | tctgtcgctc tgttggtcat cttggcctgt gtgttatgga aaaaaaggat taagcctatc |
| 901 | gtatggccca gtctccccga tcataagaag actctggaac atctttgtaa gaaaccaaga |
| 961 | aaaaatttaa atgtgagttt caatcctgaa agtttcctgg actgccagat tcatagggtg |
| 1021 | gatgacattc aagctagaga tgaagtggaa ggttttctgc aagatacgtt tcctcagcaa |
| 1081 | ctagaagaat ctgagaagca gaggcttgga ggggatgtgc agagccccaa ctgcccatct |
| 1141 | gaggatgtag tcatcactcc agaaagcttt ggaagagatt catccctcac atgcctggct |
| 1201 | gggaatgtca gtgcatgtga cgcccctatt ctctcctctt ccaggtccct agactgcagg |
| 1261 | gagagtggca agaatgggcc tcatgtgtac caggacctcc tgcttagcct tgggactaca |
| 1321 | aacagcacgc tgccccctcc attttctctc caatctggaa tcctgacatt gaacccagtt |
| 1381 | gctcagggtc agcccattct tacttccctg ggatcaaatc aagaagaagc atatgtcacc |
| 1441 | atgtccagct tctaccaaaa ccagtgaagt gtaagaaacc cagactgaac ttaccgtgag |
| 1501 | cgacaaagat gatttaaaag ggaagtctag agttcctagt ctccctcaca gcacagagaa |
| 1561 | gacaaaatta gcaaaacccc actacacagt ctgcaagatt ctgaaacatt gctttgacca |
| 1621 | ctcttcctga gttcagtggc actcaacatg agtcaagagc atcctgcttc taccatgtgg |
| 1681 | atttggtcac aaggtttaag gtgacccaat gattcagcta tttaaaaaaa aaagaggaaa |
| 1741 | gaatgaaaga gtaaaggaaa tgattgagga gtgaggaagg caggaagaga gcatgagagg |
| 1801 | aaagaaagaa aggaaaataa aaaatgatag ttgccattat taggatttaa tatatatcca |
| 1861 | gtgctttgca agtgctctgc gcaccttgtc tcactccatc ctgacaataa tcctgggagg |
| 1921 | tgtgtgcaat tactacgact actctctttt ttatagatca ttaaattcag aactaaggag |
| 1981 | ttaagtaact tgtccaagtt gttcacacag tgaagggagg ggccaagata tgatggctgg |
| 2041 | gagtctaatt gcagttccct gagccatgtg cctttctctt cactgaggac tgccccattc |
| 2101 | ttgagtgcca aacgtcacta gtaacagggt gtgcctagat aatttatgat ccaaactgag |
| 2161 | tcagtttgga aagtgaaagg gaaacttaca tataatccct ccgggacaat gagcaaaaac |
| 2221 | taggactgtc cccagacaaa tgtgaacata catatcatca cttaaattaa aatggctatg |
| 2281 | agaaagaaag agggggagaa acagtcttgc gggtgtgaag tcccatgacc agccatgtca |
| 2341 | aaagaaggta aagaagtcaa gaaaaagcca tgaagcccat ttggtttcat ttttctgaaa |
| 2401 | ataggctcaa gagggaataa attagaaact cacaatttct cttgtttgtt accaagacag |
| 2461 | tgattctctt gctgctacca cccaactgca tccgtccatg atctcagagg aaactgtcgc |
| 2521 | tgaccctgga catgggtacg tttgacgagt gagaggaggc atgacccctc ccatgtgtat |
| 2581 | agacactacc ccaacctaaa ttcatcccta aattgtccca agttctccag caatagaggc |
| 2641 | tgccacaaac ttcagggaga aagagttaca agtacatgca atgagtgaac tgactgtggc |
| 2701 | tacaatcttg aagatatacg gaagagacgt attattaatg cttgacatat atcatcttgc |
| 2761 | ctttcttggt ctagactgac ttctaatgac taactcaaag tcaaggcaac tgagtaatgt |
| 2821 | cagctcagca aagtgcagca aacccatctc ccacaggcct ccaaaccctg gctgttcaca |
| 2881 | gaaccacaaa gggcagatgc tgcacagaaa actagagaag gggtcatagg ttcatggttt |
| 2941 | tgtttgagat ttgttgctac tgtttttctg ttttgaattt tcttctttgt tctgttttta |
| 3001 | ctttatttag ggggactagg tgtttctgat attttagttt tcttgtttgt tttgttttgt |
| 3061 | gttgtctgtg aatggggttt taactgtgga tgaatggacc ttatctgttg gcttaaagga |
| 3121 | ctggtaagat cagaccatct tattcttcag gtgaatgttt tactttccaa agtgctctcc |
| 3181 | tctgcaccag cagtaataaa tacaatgcca taatccctta ggtttgccta gtgcttttgc |
| 3241 | aattttcaaa gcacttccat aagcattcct tccacctcct tgataggcat ttatggaaag |
| 3301 | cctgctacat gtcaatcata ctgttaggca caggggacct aaagacacat aaaaggatgg |
| 3361 | cattctgcct cataaattgc aaaacctaat gaaagtgact gcttggtaaa caaattatta |
| 3421 | ttatattata aaatgctata aaagagccat attgaaagtg ccctgttgga gacagggcaa |
| 3481 | atgccacaaa aatgatgtaa atttacatgg aggaaaagta gaatctgcct ggtttgtagg |
| 3541 | cagcagaaga catttttcat cagtgggcag gtgttcttta ccttttgtag aaatgggagt |
| 3601 | caagtctcaa ataggaggct ccacaaaatc tcatgccagg tctctgatac cttattcaca |
| 3661 | gaagttcttt gaagtattta ttgttatttt ctttgactta tgggaaaact gggacacagg |
| 3721 | aagacaggta aattacccaa cctcacacgt taagtcagaa ctgggagcca taattttgta |
| 3781 | tccctggtat aaatagacaa tctcttgaag aaatgaagag atgaccatag aaaaacatcg |
| 3841 | agatatctcc agctctaaaa tcctttgttt caatgttgtt tggcatatgt tatctttgga |
| 3901 | atttagtgtc tgagcctctg tctgttactg tagtatttaa aatgcatgta ttataatcat |
| 3961 | ataatcataa ctgctgttaa ttcttgatta tatacctagg gacaatgtgt aatgtaagat |
| 4021 | tactaattgg ttctgcccaa tctcctttca gattttatta ggaaaaaaaa ataaacctcc |
| 4081 | tgatcggaga caatgtatta atcagaagtg taaactgcca gttctatata gcatgaaatg |
| 4141 | aaaagacagc taatttggtc caacaaacat gactgggtct agggcaccca ggctgattca |
| 4201 | gctgatttcc taccagcctt tgcctcttcc ttcaatgtgg tttccatggg aatttgcttc |
| 4261 | agaaaagcca agtatgggct gttcagaggt gcacacctgc attttcttag ctcttctaga |
| 4321 | ggggctaaga gacttggtac gggccaggaa gaatatgtgg cagagctcct ggaaatgatg |
| 4381 | cagattaggt ggcatttttg tcagctctgt ggtttattgt tgggactatt ctttaaaata |
| 4441 | tccattgttc actacagtga agatctctga tttaaccgtg tactatccac atgcattaca |
| 4501 | aacatttcgc agagctgctt agtatataag cgtacaatgt atgtaataac catctcatat |
| 4561 | ttaattaaat ggtatagaag aaca |

Nucleotides 88-1467 translate into the above amino acid sequence and also encodes for IL-7R. In certain embodiments, the nucleotide sequence encoding human IL-7R comprises nucleotides 88-1467. In certain embodiments, the nucleotide sequence encoding human IL-7R (inclusive of part of 5′UTR) comprises nucleotides 1-1467. In certain embodiments, the nucleotide sequence encoding human IL-7R comprises the nucleotide sequence provided in FIG. 6. In certain embodiments, the nucleotide sequence encoding human IL-7R further comprises at least part of or all of the 5′UTR of the IL-7R gene (e.g., including the promoter). The at least part of the 5′UTR may comprise or overlap with DHS1 and/or DHS2.

In certain embodiments, the amino acid or nucleotide sequence of IL-7R has at least 80, 81, 82, 83, 84, 85, 86, 87, 88, 89, 90, 91, 92, 93, 94, 95, 96, 97, 98, or 99% identity with either of the above sequences. In certain embodiments, the nucleic acid molecule encoding the IL-7R is codon optimized and/or codon modified from the wild-type nucleotide sequence.

Second, in certain embodiments of the instant invention, the enhancer is an enhancer from the IL-7R gene (e.g., from the 5′UTR). In certain embodiments, the enhancer comprises at least one DNA accessibility/hypersensitivity (DHS) region (e.g., from the IL-7R gene (e.g., from the 5′UTR)). In certain embodiments, the enhancer comprises DHS1. In certain embodiments, the enhancer comprises DHS2. In certain embodiments, the enhancer comprises DHS1 and DHS2 (e.g., wherein DHS2 is 5′ to DHS1). In certain embodiments, the nucleotide sequence of DHS2 comprises:

(SEQ ID NO: 3)
CTGCAGGGAATATCCAGGAGGAACAATAATTTCAGAGGCTCTGTCTCTTC
ATGTCCTTGACCTCTGCTTACAGCAGCAATACTTTTACTCAGACTTCCTG
TTTCTGGAACTTGCCTTCTTTTTTGCTGTGTTTATACTTCCCTTGTCTGT
GGTTAGATAAGTATAAAGCCCTAGATCTAAGCTTCTCTGT.

In certain embodiments, the nucleotide sequence of DHS1 comprises:

(SEQ ID NO: 4)
CTTCCTCCCTCCCTCCCTTCCTCTTACTCTCATTCATTTCATACACACTG
GCTCACACATCTACTCTCTCTCTCTATCTCTCTCAGA.

In certain embodiments, the nucleotide sequence of DHS1 or DHS2 has at least 80, 81, 82, 83, 84, 85, 86, 87, 88, 89, 90, 91, 92, 93, 94, 95, 96, 97, 98, or 99% identity with either of the above sequences.

Third, in certain embodiments of the instant invention, the promoter is the PGK promoter. In certain embodiments, the PGK promoter is human. In certain embodiments, the nucleotide sequence of the PGK promoter comprises the nucleotide sequence provided in FIG. 6. In certain embodiment, the nucleotide sequence of the PGK promoter is from GenBank Accession No. NG_008862. In certain embodiments, the nucleotide sequence of the PGK promoter comprises:

(SEQ ID NO: 5)
CTCGAATTCCACGGGGTTGGGGTTGCGCCTTTTCCAAGGCAGCCCTGGGT
TTGCGCAGGGACGCGGCTGCTCTGGGCGTGGTTCCGGGAAACGCAGCGGC
GCCGACCCTGGGTCTCGCACATTCTTCACGTCCGTTCGCAGCGTCACCCG
GATCTTCGCCGCTACCCTTGTGGGCCCCCCGGCGACGCTTCCTGCTCCGC
CCCTAAGTCGGGAAGGTTCCTTGCGGTTCGCGGCGTGCCGGACGTGACAA
ACGGAAGCCGCACGTCTCACTAGTACCCTCGCAGACGGACAGCGCCAGGG
AGCAATGGCAGCGCGCCGACCGCGATGGGCTGTGGCCAATAGCGGCTGCT
CAGCGGGGCGCGCCGAGAGCAGCGGCCGGGAAGGGGCGGTGCGGGAGGCG
GGGTGTGGGGCGGTAGTGTGGGCCCTGTTCCTGCCCGCGCGGTGTTCCGC
ATTCTGCAAGCCTCCGGAGCGCACGTCGGCAGTCGGCTCCCTCGTTGACC
GAATCACCGACCTCTCTCCCCAG.

In certain embodiments, the nucleotide sequence of the PGK promoter has at least 80, 81, 82, 83, 84, 85, 86, 87, 88, 89, 90, 91, 92, 93, 94, 95, 96, 97, 98, or 99% identity with the above sequence.

Fourth, the Woodchuck Post-Regulatory Element, or WPRE can be placed outside the integrating sequence to increase the safety of the vector. In a particular embodiment, the WPRE is 3′ of the 3′LTR. The WPRE increases the titer of the lentivirus, but it can undergo chromosomal rearrangement upon integration. In order to preserve the ability of WPRE to increase viral titers without having this viral element in the integrating sequence, the WPRE ca be removed from the integrating portion and added, for example, after the 3′LTR. In addition, a polyadenylation signal (e.g., a bovine growth hormone polyA tail) can be inserted after the WPRE region to increase lentiviral titers (Zaiss, et al. (2002) J. Virol., 76(14):7209-19). In certain embodiments, the WPRE is the Woodchuck Hepatitis Virus Posttranscriptional Regulatory Element. In certain embodiments, the WPRE comprises:

(SEQ ID NO: 6)
GTCGACAATCA ACCTCTGGAT TACAAAATTT
GTGAAAGATT GACTGGTATT CTTAACTATG TTGCTCCTTT TACGCTATGT GGATACGCTG
CTTTAATGCC TTTGTATCAT GCTATTGCTT CCCGTATGGC TTTCATTTTC TCCTCCTTGT
ATAAATCCTG GTTGCTGTCT CTTTATGAGG AGTTGTGGCC CGTTGTCAGG CAACGTGGCG
TGGTGTGCAC TGTGTTTGCT GACGCAACCC CCACTGGTTG GGGCATTGCC ACCACCTGTC
AGCTCCTTTC CGGGACTTTC GCTTTCCCCC TCCCTATTGC CACGGCGGAA CTCATCGCCG
CCTGCCTTGC CCGCTGCTGG ACAGGGGCTC GGCTGTTGGG CACTGACAAT TCCGTGGTGT
TGTCGGGGAA GCTGACGTCC TTTCCATGGC TGCTCGCCTG TGTTGCCACC TGGATTCTGC
GCGGGACGTC CTTCTGCTAC GTCCCTTCGG CCCTCAATCC AGCGGACCTT CCTTCCCGCG
GCCTGCTGCC GGCTCTGCGG CCTCTTCCGC GTCTTCGCCT TCGCCCTCAG ACGAGTCGGA
TCTCCCTTTG GGCCGCCTCC CCGCCTGGAA TTCGAGCTCG GTACC,

or the complement thereof. In certain embodiments, the nucleotide sequence of the WPRE has at least 80, 81, 82, 83, 84, 85, 86, 87, 88, 89, 90, 91, 92, 93, 94, 95, 96, 97, 98, or 99% identity with any of the above sequences.

Fifth, in certain embodiments of the instant invention, the vector may comprise insulators to maximize IL-7R expression at a random site of integration and to protect the host genome from possible genotoxicity. Insulators can shelter the transgenic cassette from the silencing effect of non-permissive chromatin sites and, at the same time, protect the genomic environment from the enhancer effect mediated by active regulatory elements introduced with the vector. The 1.2 Kb cHS4 insulator has been used to rescue the phenotype of thalassemic CD34+BM-derived cells (Puthenveetil, et al. (2004) Blood, 104(12):3445-53). Further, fetal hemoglobin can be synthesized in human CD34⁺-derived cells after treatment with a lentiviral vector encoding the gamma-globin gene, either in association with the 400 bp core of the cHS4 insulator or with a lentiviral vector carrying an shRNA targeting the gamma-globin gene repressor protein BCL 11A (Wilber, et al. (2011) Blood, 117(10):2817-26). The HS2 enhancer of the GATA1 gene has also been used to achieve high beta-globin gene expression in human cells from patients with beta-thalassemia (Miccio, et al. (2011) PLOS One, 6 (12): e27955). The use of a 200 bp insulator, derived from the promoter of the ankyrin gene, resulted in a significant amelioration of the thalassemic phenotype in mice and high level of expression was reached in both human thalassemic and SCD cells (Breda, et al. (2012) PloS one 7(3):e32345). In certain embodiments, the ankyrin insulator comprises:

(SEQ ID NO: 7)
gtgc gggccaggcc cccgagggcc ttatcggccc cagaggcgct
tgctgtcggg ccgggcgctc ccggcacggg cgggcggagg
ggtggcgccc gcctggggac cgcagattac aagagcacct
cctcccccaa ccccaggagg ccccgctccc caggcctcgg
ccggcgcgga cccctggttg ccccgg,

or the complement thereof. The foamy virus has a 36-bp insulator located in its long terminal repeat (LTR) which reduces its genotoxic potential (Goodman, et al. (2018) J. Virol., 92: e01639-17). In certain embodiments, the foamy virus insulator comprises AAGGGAGACATCTAGTGATATAAGTGTGAACTACAC (SEQ ID NO: 8) of the complement thereof. In certain embodiments, the insulator sequence has at least 80, 81, 82, 83, 84, 85, 86, 87, 88, 89, 90, 91, 92, 93, 94, 95, 96, 97, 98, or 99% identity with any of the above sequence.

In certain embodiment, the viral vector of the instant invention has a nucleotide sequence identical to those presented herein or they can have least 80, 81, 82, 83, 84, 85, 86, 87, 88, 89, 90, 91, 92, 93, 94, 95, 96, 97, 98, or 99% identity to the nucleotide sequence of a viral vector disclosed herein or to an element of a nucleotide sequence of a viral vector disclosed herein (e.g., the sequences provided herein (e.g., FIGS. 6 and 7) and in U.S. Patent Application Publication 2018/0008725; WO 2019/213011; and WO 2020/264488) or the complement thereof.

The present disclosure provides compositions and methods for the inhibition, prevention, and/or treatment of IL-7R deficiency (e.g., IL-7R SCID). Methods of increasing IL-7R expression and/or production are also provided. In certain embodiments, the methods comprise introducing the vectors or nucleic acids of the instant invention into a cell.

In accordance with another aspect of the instant invention, methods of transducing cells with a viral vector of the instant invention are provided. In a particular embodiment, the transduction is performed with an adjuvant/enhancer such as LentiBoost™ or cyclosporine H. In certain embodiments, the viral vector is pseudotyped with Cocal envelope. In certain embodiments, the viral vector is pseudotyped with VSV-G.

In accordance with the instant invention, compositions and methods are provided for increasing IL-7R production in a cell or subject. The method comprises administering a viral vector of the instant invention to the cell, particularly a hematopoietic stem cell, bone marrow cell, or subject. In a particular embodiment, the subject has a IL-7R deficiency. In a particular embodiment, the subject has IL-7R SCID. The viral vector may be administered in a composition further comprising at least one pharmaceutically acceptable carrier.

In accordance with another aspect of the instant invention, compositions and methods for inhibiting (e.g., reducing or slowing), treating, and/or preventing an IL-7R deficiency (e.g., IL-7R SCID) in a subject are provided. In certain embodiments, the methods comprise administering to a subject in need thereof a viral vector of the instant invention. The viral vector may be administered in a composition further comprising at least one pharmaceutically acceptable carrier. The viral vector may be administered via an ex vivo methods wherein the viral vector is delivered to a hematopoietic stem cell or bone marrow cell, particularly autologous ones, and then the cells are administered to the subject. In a particular embodiment, the method comprises isolating hematopoietic stem cells or bone marrow cells from a subject, delivering a viral vector of the instant invention to the cells, and administering the treated cells to the subject. The methods of the instant invention may further comprise monitoring the disease or disorder in the subject after administration of the composition(s) of the instant invention to monitor the efficacy of the method. For example, the subject may be monitored for IL-7R levels which may be compared to previous or other samples from the subject or to a standard.

The methods of the instant invention may further comprise the administration of another therapeutic regimen for treating IL-7R and/or its symptoms.

As explained hereinabove, the compositions of the instant invention are useful for increasing IL-7R production and for treating n IL-7R deficiency. A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intrarectal, intramuscular, and intranasal administration. In a particular embodiment, the composition is administered directly to the blood stream (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, PA. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from a disease or disorder, including improvement in the condition of the patient (e.g., in one or more symptoms), delay in the progression of the condition, etc.

As used herein, the term “prevent” refers to the prophylactic treatment of a subject who is at risk of developing a condition and/or sustaining a disease or disorder, resulting in a decrease in the probability that the subject will develop conditions associated with the IL-7R deficiency.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with an IL-7R deficiency.

As used herein, the term “subject” refers to an animal, particularly a mammal, particularly a human.

A “vector” is a genetic element, such as a plasmid, cosmid, bacmid, phage or virus, to which another genetic sequence or element (either DNA or RNA) may be attached so as to bring about the replication and/or expression of the attached sequence or element. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism.

The following example is provided to illustrate various embodiments of the present invention. It is not intended to limit the invention in any way.

### Example

Five vectors were generated and confirmed by restriction enzyme digestion. These vectors are depicted in FIG. 1 (top to bottom): vPGK_GFP; vPGK_hIL7R; vPGK_DHS1_hIL7R; vDHS1_hIL7R; and vDHS2DHS1_hIL7R.

To test the expression of human IL-7R, the Jurkat cell line was used. Jurkat cell line is an immortalized line of human T lymphocyte cells derived from a human T-cell acute lymphocytic leukemia (T-ALL) specimen. Jurkat cells express minimal IL-7R, but do express CD132 (IL-2R common gamma chain) and STAT5 (phosphorylation target of IL-7, showing IL-7R signaling). IL7-R expression in the Jurkat cells was knocked out using CRISPR and sgRNA targeting exon 2. Expression of IL-7R was studied using anti-IL-7R (CD127) antibodies and flow cytometry. Notably, treating T cells with 5-aza-2′-deoxycytidine, which reduces DNA methylation, increased IL-7R expression, thereby indicating that methylation of the promoter or 5′UTR can effect IL-7R expression.

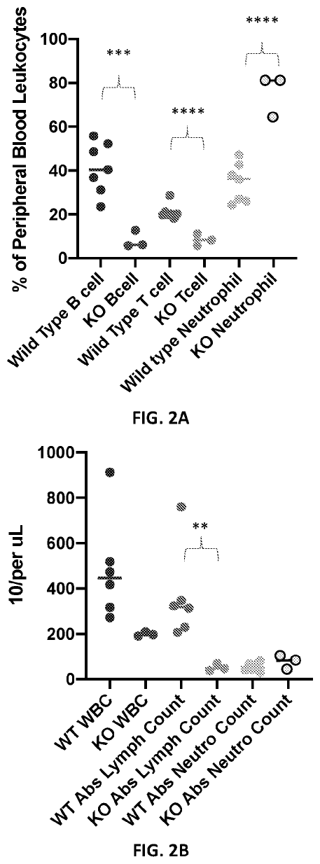

Mouse knockouts (KO) of IL7R have T-B immunodeficiency and are commercially available. As seen in FIG. 2A, T and B cell reduction results in increase in relative size of the neutrophil compartment. As seen in FIG. 2B, the absolute lymphocyte count (ALC) is lower in IL-7R KO mice. Specifically, IL-7R have an 86% reduction in ALC.

Transduction of Il7r^{-/-} bone marrow cells with IL7R encoding VSV-G pseudotyped lentiviral vectors (LV) rescued the formation of lymphocyte precursors from murine bone marrow cells in colony forming unit (CFU) assays (pre-B CFU with human IL-7), with the most robust response seen with vPGK_DHS_hIL7R although the response with vDHS1 was similar. Briefly, CD45.2 Il7r^{-/-} mice were used or CD45.1 controls. Mouse bone marrow (300-400k cells) from Il7r^{-/-} animals were transduced ex vivo. A single dose of vector was used with a multiplicity of infection (MOI) 50 was used for vPGK_DHS1 and a MOI 37.5 was used for vDHS1. The MOI was based on prior in vitro transduction analysis to account for higher vector copy number (VCN). Specially, the in vitro VCN, as determined by droplet digital PCR (ddPCR) after 2 weeks, was about 4 for vPGK_DHS1 and about 6 for vDHS1. Notably, in the presence of IL-7, the VCN increased to about 6 for vPGK_DHS1 and about 8 for vDHS1. The transduced cells were then engrafted in lethally irradiated (8 Gy cesium) Il7r^{-/-} opposite gender recipients, to rapidly ascertain chimerism by sex mismatch ddPCR.

There were no significant aberrations in absolute neutrophil count, hemoglobin, or platelet count. Further, there was no evidence of leukemia after 2 months.

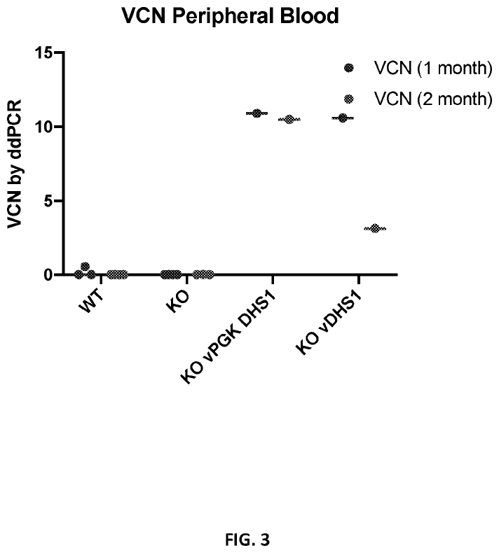

The VCN of peripheral blood cells was measured at 1 and 2 months post transplant. As seen in FIG. 3, the VCN for vPGK_DHS1 remained high after 2 months, but the VCN decreased in the second month for vDHS1.

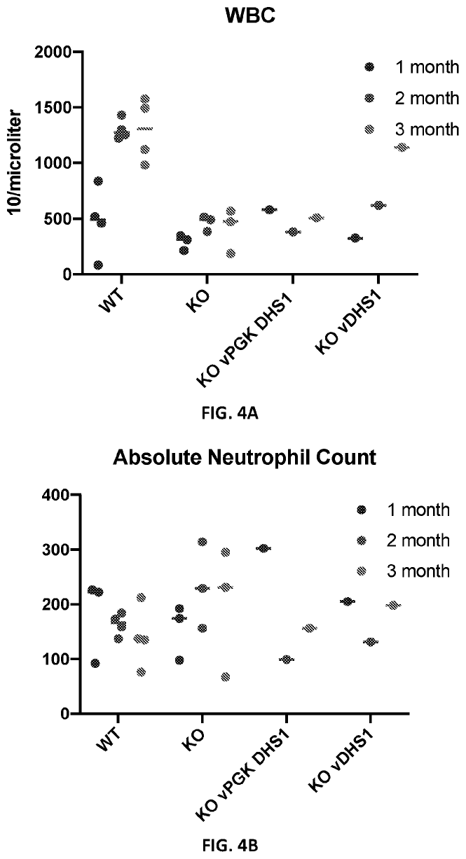

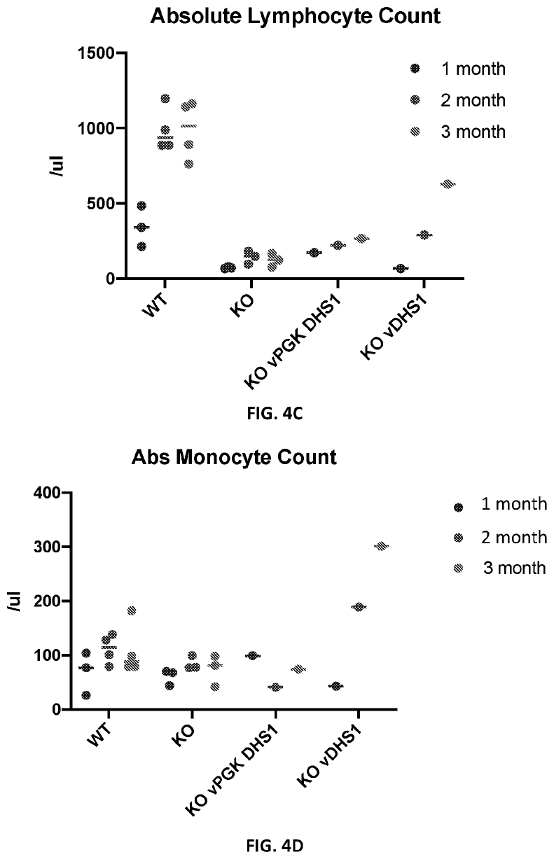

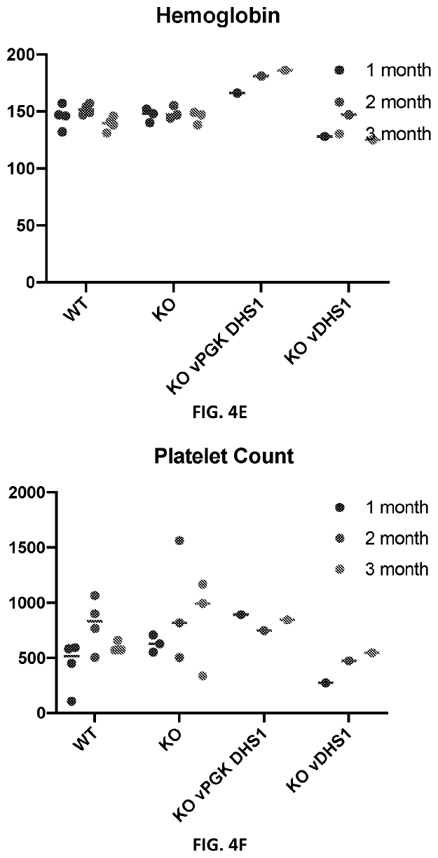

A hematology study was performed on the mice at 1, 2, and 3 months after transplant. FIGS. 4A and 4B provide the white blood cell count and absolute neutrophil count, receptively. FIGS. 4C and 4D provide the absolute lymphocyte count and absolute monocyte count, respectively. FIGS. 4E and 4F provide the hemoglobin and platelet count, respectively.

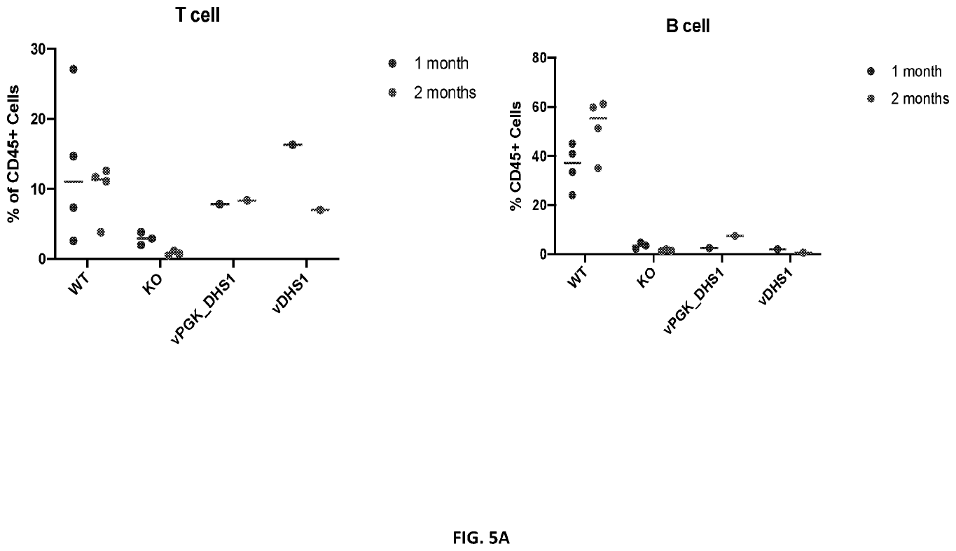

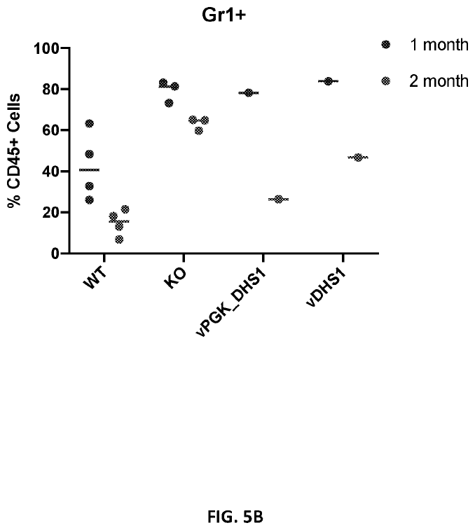

Absolute lymphocyte counts in mice receiving transduced Il7r^{-/-} bone marrow cells was higher (mean 2555/μL) than in mice receiving untransduced bone marrow (mean 1410/μL). FIG. 5A shows T-cell and B-cell reconstitution at 1 and 2 months after transplant as determined by FACS. The proportion of leukocytes that were T cells was 4.2-fold and 9.8-fold higher at 1 and 2 months post-transplant, respectively. B cells were only seen in mice receiving vPGK_DHS_hIL7R: 7.4% of leukocytes versus 1.5% in controls. A reciprocal decrease in the fraction of Gr1+ cells (neutrophils and monocytes) was seen at two months post-transplant in transduced marrow recipients compared to untransduced controls: 36.5% versus 63% Gr1+, respectively (FIG. 5B).

For individuals with IL-7R deficient SCID, but no HLA matched hematopoietic stem cell (HSC) donor, there is a difficult choice between the risks of graft-versus-host disease (GVHD) with a mismatched HSC donor and supportive care with the hope of identifying a matched HSC donor in the future. In immunodeficiencies, however, age and serious infection are both associated with increased mortality (Pai, et al., NJEM (2014) 371:434-446). This novel approach to IL7R gene replacement can be used as a therapeutic and expedient option for those without a matched donor. Additionally, this would be an ideal disorder for hematopoietic stem cells (HSC) conditioning with less toxic, HSC-targeted strategies given gene corrected lymphocytes and progenitors will preferentially expand post-transplant.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A lentiviral vector comprising a nucleic acid molecule comprising:
i) a 5′ long terminal repeat (LTR) and a 3′ LTR;
ii) a nucleic acid sequence encoding a human IL-7R, optionally including at least part of the 5′UTR of the IL-7R gene.

2. The lentiviral vector of claim 1, wherein said at least part of the 5′UTR comprises the IL-7R promoter and/or an IL-7R enhancer element.

3. The lentiviral vector of claim 2, wherein said IL-7R enhancer element is DHS1 and/or DHS2.

4. The lentiviral vector of claim 1, wherein at least one of said LTR is self-inactivating.

5. The lentiviral vector of claim 1, further comprising a polyadenylation signal.

6. The lentiviral vector of claim 1, further comprising a Woodchuck Post-Regulatory Element (WPRE).

7. The lentiviral vector of claim 1, further comprising an insulator element.

8. The lentiviral vector of claim 1, further comprising a constitutive promoter.

9. The lentiviral vector of claim 8, wherein said constitutive promoter is the phosphoglycerate kinase (PGK) promoter.

10. The lentiviral vector of claim 1, further comprising DHS1 and the phosphoglycerate kinase (PGK) promoter.

11. A composition comprising the lentiviral vector of claim 1 and a pharmaceutically acceptable carrier.

12. A composition comprising viral particles, wherein the viral particles comprise the lentiviral vector of claim 1.

13. The lentiviral vector of claim 1, wherein the lentiviral vector is present in a cell.

14. The lentiviral vector of claim 13, wherein the cells have been isolated from an individual with an IL-7R deficiency.

15. A method of inhibiting, treating, and/or preventing an IL-7R deficiency in a subject, said method comprising administering the lentiviral vector of claim 1 to the subject.

16. The method of claim 15, wherein the hematopoietic stem cells or bone marrow cells are isolated from the subject to be treated.

17. The method of claim 15, wherein said subject has IL-7R SCID.

18. A method of increasing expression of IL-7R, said method comprising delivering the lentiviral vector of claim 1 to a cell.

19. A method of inhibiting, treating, and/or preventing an IL-7R deficiency in a subject, said method comprising introducing the lentiviral vector of claim 1 into hematopoietic stem cells or bone marrow cells and delivering the hematopoietic stem cells or bone marrow cells to the subject.
